(19) United States
(12) Patent Application Publication
(10) Pub. No.: US 2010/0241169 A1
(43) Pub. Date: Sep. 23, 2010

(54) LINKING ELEMENT OF A VERTEBRAL OSTEOSYNTHESIS DEVICE AND VERTEBRAL OSTEOSYNTHESIS DEVICE COMPROMISING IT

(75) Inventors: Mingyan Liu, Bourg-la-Reine (FR); Loic Josse, Denens (CH)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/407,601

(22) Filed: Mar. 19, 2009

(51) Int. Cl. A61B 17/70

(52) U.S. Cl. 606/263; 606/264; 606/279

(57) ABSTRACT

A linking element between a longitudinal element of a vertebral osteosynthesis device and a vertebra that comprises a left part and a right part, each intended to extend on a respective side of the spinous process of the vertebra. The left part and the right part each delimit a spinous surface and a laminary surface, and include a tightening means to bring them closer to each other, and tightens them on the spinous process by integrally connecting them to each other. The left part and the right part each delimit a reception recess for the longitudinal element and include immobilization means for the linking element assembled on the vertebra.

An osteosynthesis device comprising at least one such linking element.

### BACKGROUND OF THE INVENTION

The present invention concerns the field of prostheses called “vertebral osteosynthesis devices” and is intended to be implanted on the spinal column of a patient.

Various pathologies may require the implantation of such a device, such as damage to the vertebrae and/or the disks that separate them, as a result of a degenerative disease, a tumor or a trauma, or a misalignment of the vertebrae with respect to their normal position. Their purpose is to integrally connect two or more consecutive vertebrae in a predetermined relative position. For this purpose, one often uses longitudinal elements, such as rods or elongated plates that extend along the spinal column and are fixed to the vertebrae by various means.

These longitudinal elements can be fixed to the column using an anterior surgical approach or a posterior surgical approach. The two approaches have advantages and disadvantages, and the choice of method by the surgeon must be made as a function of the particular needs of the patient. The anterior approach may be particularly indicated when the vertebrae of a patient have a nonpathological internal structure, for example, an absence of osteoporosis affecting the spongy bone, which makes possible the implantation of bone screws to which the longitudinal elements are connected (directly or through the intermediary of connectors).

The posterior approach may be more indicated in the case where osteoporosis weakens the spongy part of the vertebrae, and makes the implantation of bone screws risky in regions where the spongy bone is particularly thick, as is the case in the anterior part of the vertebrae. In this case, one can use the vertebral processes to implant, on the posterior part of the vertebrae, hooks that engage on these processes, where these hooks themselves carry elements that allow the connection of a longitudinal element to them.

One object of the invention is to propose a device for the fixation of a longitudinal element by the posterior route, the design of which is simple and the positioning easy, requiring as short an operating time as possible.

### SUMMARY OF THE INVENTION

To this effect, the present invention relates to a linking element for linking a longitudinal element of a vertebral osteosynthesis device with a vertebra, that is characterized in that it comprises a left linking part and a right linking part, each intended to extend on the left side and on the right side, respectively, of the spinous process of said vertebra. The left part and the right part each delimits a spinous surface that is shaped to rest on the spinous process, and includes a laminary surface that is shaped to rest on the posterior surface of the lamina of said vertebra.

The left part and the right part comprise tightening means to bring the left part and the right part closer to each other, and to tighten the left part and the right part on the spinous process by integrally connecting the left part and the right part,

A least one of the left part and the right part delimits a recess for receiving the longitudinal element, and where the linking element comprises means for keeping the longitudinal element in said recess.

At least one of the left part and of the right part comprises means to immobilize the linking element assembled on the vertebra.

According to special embodiments of the invention, the linking element comprises one or more of the following characteristics, which are taken separately or in any of the technically possible combinations:

the tightening means comprises locking devices provided on the left part and on the right part;

the tightening means comprises a first pair and a second pair of locking devices;

the first locking means pair is arranged in proximity to the inferior side of the spinous process, and the second locking means pair is arranged in proximity to the superior side of the spinous process;

the first pair ensures an immobilization of the left part with the right part in translation along three mutually orthogonal directions, where the second pair ensures an immobilization of the left part with the right part with a degree of freedom in translation;

the first pair comprises a serrated pin that has a rotational symmetry about its axis of insertion (B) in a corresponding orifice, where the second pair comprises a toothed tongue which is adapted for insertion into a corresponding clip;

the immobilization means comprises an infra-laminary hook that is intended to become anchored to the infra-laminar part of said vertebra;

the immobilization means comprises a laminary cutting edge, which is intended to become anchored to the cephalad part of the spinous process of the vertebra;

the element comprises at least one bone growth orifice that opens opposite said vertebra and that preferably traverses, preferably a plurality of osseous bone orifices distributed over the entire left part and right part;

the element comprises at least one penetrating edge, which is intended to penetrate into said vertebra;

the element comprises at least one pair of penetrating edges that define a bone growth furrow between them; and

the laminary surface is formed by a plurality of flexible legs.

An object of the invention is a vertebral osteosynthesis device comprising at least one longitudinal element and linking means for linking said longitudinal element to at least two vertebrae, characterized in that it comprises at least one linking element of the type according to any one of the preceding claims.

According to another embodiment, the vertebral osteosynthesis device comprises a left longitudinal element and a right longitudinal element extending respectively from the left side and from the right side of the spinous process of the vertebra, where said left part receives the left longitudinal element, and said right part receives the right longitudinal element.

### BRIEF DESCRIPTION OF THE FIGURES

The invention may be better understood upon reading the following description, which is given only as an example, and is made in reference to the drawings in the appendix, in which:

FIG. 1 is an oblique view of an osteosynthesis device according to the invention, implanted on a part of the spinal column of a patient;

FIG. 2 is a posterior oblique exploded view of a linking element of the osteosynthesis device of FIG. 1;

FIG. 3 is an anterior oblique exploded view of a part of the osteosynthesis device of FIG. 1, illustrating the linking element of FIG. 2;

FIG. 4 is a schematic cross-sectional view along the plane IV of FIG. 1, of a linking element arranged on a vertebra; and

FIG. 5 is a lateral oblique view of a linking element according to a second embodiment of the invention.

### DESCRIPTION OF THE ILLUSTRATED EMBODIMENTS

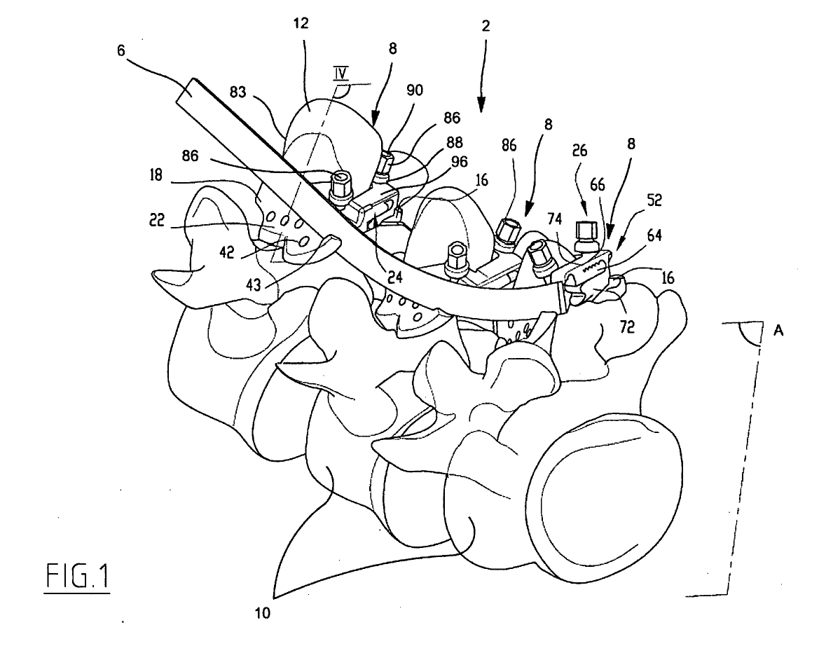

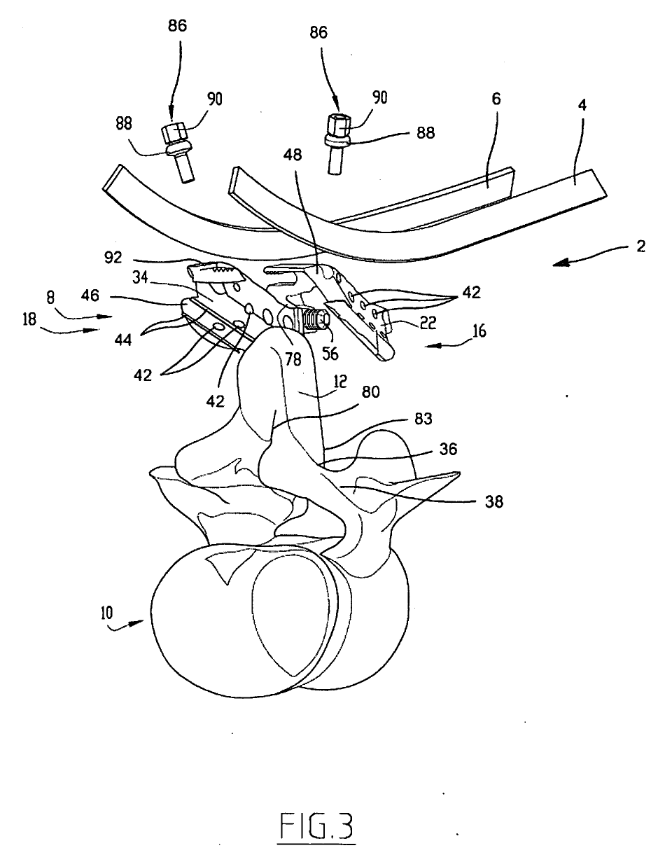

FIG. 1 illustrates a vertebral osteosynthesis device 2 for the posterior route comprising two longitudinal elements, a left longitudinal element 4 and a right longitudinal element 6 (for greater clarity only the right one is represented in FIG. 1; the two longitudinal elements 4, 6 are visible in FIG. 3), and three linking elements 8 between the longitudinal elements 4, 6 and three successive vertebrae 10. In the example illustrated, the vertebrae are L3, L4, and L5. However, the device 2 could also be inserted on the thoracic vertebrae and/or other lumbar vertebrae.

In the entire description, the terms “inferior,” “superior,” “posterior,” and “anterior” are understood to be with respect to the vertebrae 10. They are understood to refer to the standing position of the patient. The terms “left” and “right” are understood to be with respect to the patient seen from the back, which corresponds to the view of the patient's body during the posterior approach.

The left longitudinal element 4 and the right longitudinal element 6 extend substantially symmetrically on both sides of the spinous process 12 of the vertebra 10.

The three linking elements 8 are identical in the example represented, and only one of them will be described below.

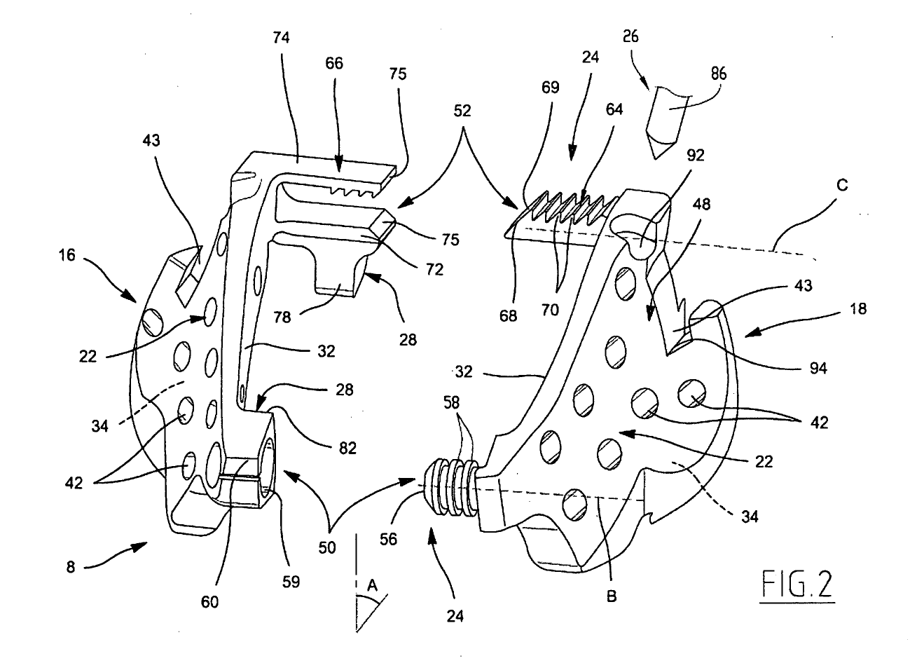

As illustrated in greater detail in FIG. 2, each linking element 8 comprises a left linking part 16 and a right linking part 18, each intended to extend, respectively, on the left side and on the right side of the spinous process 12 of the corresponding vertebra 10.

Each left part 16 and right part 18 comprises a curved body 22 supported on the vertebra 10, and tightening and integrally connecting means 24 to bring the left part 16 and the right part 18 closer to each other, and to connect them integrally by “sandwiching” the spinous process 12.

Each part 16, 18 also comprises holding means 26 for the corresponding longitudinal element 4 and immobilization means 28 for the linking element 8 assembled on the vertebra 10.

The curved bodies 22 of the left part 16 and of the right part 18 are symmetric with respect to a median vertical plane A of the vertebra 10, and only the body 22 of the right part 18 will be described below.

The support body 22 preferably has, as represented, a spinous surface 32 which is shaped to rest on the spinous process 12, and a laminary surface 34 which is shaped to rest on the posterior surface 36 (or “external”) of the lamina 38 of the vertebra 10, at the level of the posterior arch (see FIGS. 2 and 3).

The body 22 presents a plurality of bone growth orifices 42 opening onto the spinous surface 32 and onto the laminary surface 34.

The body 22 delimits in addition a reception recess 43 for the longitudinal element 4.

The spinous surface 32 is smooth and of generally planar shape, but it may have a cuneiform shape in adaptation to the shape of the spinous process 12.

As is visible in FIG. 3, the laminary surface 34 is curved substantially so as to conform to the shape of the posterior surface 36 of the lamina 38 of the vertebra 10.

The laminary surface 34 defines two penetrating edges 44 that are intended to penetrate the vertebra 10.

The penetrating edges 44 define between them a furrow 46 for bone growth, which is intended to receive the forming bone material.

The growth orifices 42 extend over the entire spinous surface 32 and laminary surface 34. In the example illustrated, they are distributed regularly over the body 22.

The growth orifices 42 pass through, and allow the possible insertion of bone grafts. They are cylindrical, of circular section, but they may be of any suitable shape.

The diameter of the orifices 42 is, for example, between 1 mm and 5 mm.

Preferably, at least one of the orifices 42 opens out into the bone growth furrow 46.

The reception recess 43 (see FIG. 2) is a notch having a shape complementary to the longitudinal element 4. The recess 43 is inclined with respect to the median plane A, so that the longitudinal element 4 is inclined with respect to the plane A, but it can be parallel to the plane A according to a special configuration.

The reception recess 43 is provided in the upper part 48 of the body 22. Its shape can vary as a function of the cross section of the longitudinal elements 4 and 6, and it can be, for example, rectangular, ovoid, circular, etc.

The mutual tightening and integrally connecting means 24 for the left part and right part, 16 and 18, comprise two pairs, an inferior pair 50 and a superior pair 52, of locking devices (see FIG. 2), where the inferior pair 50 is arranged in proximity to the lower side of the spinous process 12, and the superior pair 52 is arranged in proximity to the superior side of the spinous process 12.

The lower pair 50 comprises a pin 56 that has rotational symmetry about its insertion axis B. The pin 56 has a plurality of annular locking ribs 58.

A complementary locking orifice 59 is provided on a protuberance 60 of the left part 16.

The connection between the pin 56 and the orifice 59 immobilizes the left part 16 and the right part 18 with respect to each other in translation along three orthogonal directions. It allows, in addition, a mutual rotation of the left part and the right part, 16 and 18, about the insertion axis B of the pin 56.

The superior pair 52 comprises a toothed tongue 64, which is provided on the right part 18, and a complementary locking clip 66, which is provided on the left part 16.

The toothed tongue 64 is adapted for insertion along an axis C that is parallel to the insertion axis B of the pin 56.

The tongue 64 has a generally parallelepiped shape, and it has an insertion point 68 defining a cam surface 69 that is adapted to guide the tongue 68 into the clip 66.

The teeth 70 of the tongue 64 are shaped to allow an insertion into the clip 66, while preventing retraction from the clip 66 due to traction along the insertion axis C of the tongue 64.

The clip 66 comprises an anterior arm 72 and a posterior arm 74 forming the clip 66. The anterior arm 72 and the posterior arm 74 has surfaces complementary to the tongue 64, and two opposite cam surfaces 75 ensuring the guidance of the tongue 64 into the clip 66.

The locking connection between the tongue 64 and the clip 66 allows a degree of freedom in translation perpendicularly to the insertion axis C.

The clip 66 so formed is slightly elastic so as to deform elastically during insertion of the tongue 64.

The immobilization means 28 of the linking element 8 on the vertebra 10 can comprise an infra-laminary hook 78 intended to become anchored to the infra-laminar part 80 of the vertebra 10 (see FIG. 3) and/or a cephalad cutting edge 82 (see FIG. 2), which is intended to become anchored to the cephalad part 83 of the spinous process 12

The infra-laminary hook 78, in the represented example, is provided on the anterior arm 72 of the clip 66, and it protrudes in an anterior and inferior way with respect to this arm 72.

The holding means 26 for the element 4 comprise, for each longitudinal element 4, a holding screw 86 having a flange 88 resting on the longitudinal element, and a screw head 90 for screwing into a corresponding threaded orifice 92 provided in the body 22, at the level of the reception recess 43. In FIG. 1, the screws 86 are loose, so that their contact with the longitudinal elements 4, 6 (of which only one is represented) has not yet been made.

In the fixation position, the flange 88 rests on the longitudinal element 4, 6, and the longitudinal element 4, 6 rests on the bottom 94 of the reception recess 43.

Once inserted in the recess 43, the longitudinal element 4 is immobilized in translation along a first direction. The holding screw 86 immobilizes the longitudinal element 4 in translation along two other directions that are orthogonal to the first direction.

For the assembly of the linking element 8, the left part and the right part, 16, 18, are arranged in a posterior way on both sides of the spinous process 12, and they are brought closer to each other so as to engage partially the pin 56 in the orifice 59, and then move the tongue 64 substantially opposite the clip 66.

The surgeon then applies pressure to the left part 16 and the right part 18 to bring them even closer toward each other, in such a way as to also insert the pin 56 as deeply as possible into the orifice 59, and the tongue 64 into the clip 66, and thus produce the locking of the tightening means 24. In principle, the strength of the fingers is sufficient for this operation, however, the surgeon can naturally use an appropriate forceps.

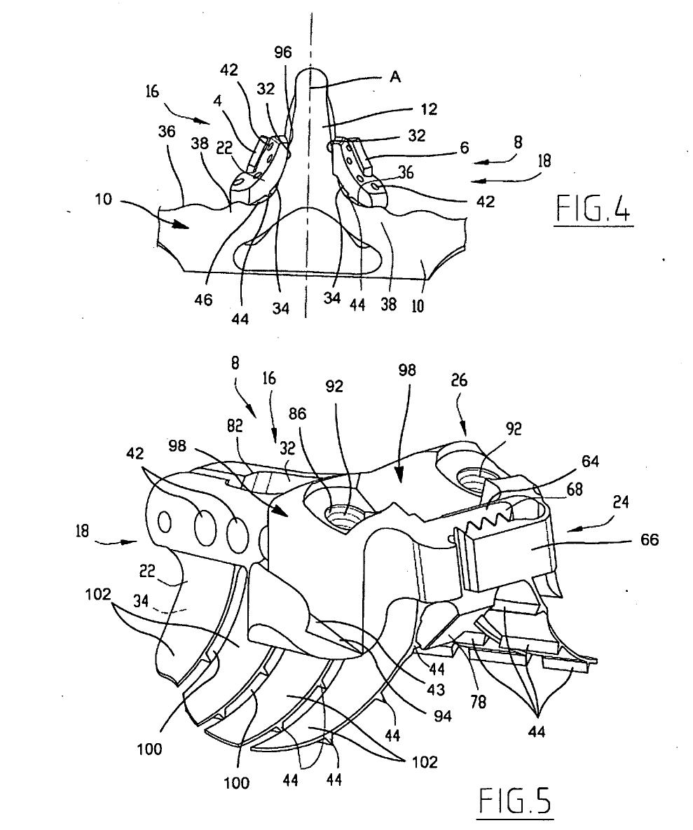

Once the linking element 8 has been assembled, the support of the spinous surface 32, of the laminary surface 34, of the infra-laminary hook 78, and of the cephalad cutting edge 82 ensure the immobilization of the linking element 8 on the vertebra 10 in three orthogonal directions. The opposite spinous surfaces 32 of the left part 16 and of the right part 18 then delimit together a through-hole reception orifice 96 (see FIG. 4) for the spinous process 12.

Once each one of the linking elements 8 is fixed to the corresponding vertebrae 10, the surgeon arranges the longitudinal elements 4 in the corresponding reception recesses 43 of the linking elements 8.

The surgeon then tightens the holding screws 86 completely in the corresponding orifice 92 of the body 22, and cuts the head 90 so that the screw 86 protrudes minimally or does not protrude at all from the posterior surface of the body 22. The head 90 is preferably adapted so as to separate from the remainder of the screw 86 above a predetermined tightening torque, which also ensures secure screwing of the longitudinal element 4, 6.

The invention thus produces an osteosynthesis device 2 that does not require the placement of screws in the vertebra 10 or the excision of the spinous process 12. The device 2 is thus not very invasive, and it presents little risk of damaging the vertebra 10.

In addition, the arrangement of the linking element 8 as two parts, a left part 16 and a right part 18, which are provided with tightening means 24, ensures a rapid and reliable fixation of the linking element 8 to the vertebra 10.

The number of pieces to the device 2 is small, which is also an advantage in terms of manufacturing cost and reliable management of the stock of manufactured and packaged elements.

In addition, the linking element 8 is adaptable to different conformations of vertebrae 10. It should be noted that, in the illustrated example, the same type of linking element 8 is fixed to three different vertebrae 10. The linking element 8 can in fact be adapted to several different conformations of vertebrae 10.

In a variant, at least one of the pairs of locking devices 50, 52 is, for example, replaced by one or more fixation screws.

FIG. 5 illustrates a linking element 8 according to a second embodiment of the invention. The identical reference numbers denote elements similar to those of the first embodiment, and only the differences with respect to the first embodiment are described below.

In this second embodiment, the body 22 comprises a posterior head 98, which protrudes, and in which are provided the reception recess 43 and the threaded reception orifice 92 for the holding screw 86. In addition, the head 98 is extended by the tongue 64 for the right part 18, and by the clip 66 for the left part 16.

The heads 98 of the left part 16 and of the right part 18 have a complementary shape.

In addition, the tongue 64 and the clip 66 extend along an insertion plane that is perpendicular to the median axis of the vertebra 10.

It should be noted that in this second embodiment, the growth orifices 42 that open onto the laminary surface 34 have been replaced by slits 100, so that the body 22 forms a plurality of distinct flexible laminar support legs 102, for the purpose of obtaining a perfect adaptation to the morphology of the posterior surface 36 of the posterior arch.

In addition, each leg 102 has a plurality of penetrating edges 44 on its laminary surface 34.

This variant has the advantage of having a better capacity for adapting the laminar surface 34 to the particular configuration of the vertebra 10 on which it is implanted.

What is claimed is:

1. A linking element between a longitudinal element of a vertebral osteosynthesis device and a vertebra, characterized in that the linking element comprises a left linking part and a right linking part wherein, each part is intended to extend on the left side and on the right side, respectively, of the spinous process of said vertebra, wherein the left part and the right part each delimit a spinous surface shaped to rest on the spinous process and a laminary surface shaped to rest on the posterior surface of the lamina of said vertebra,
wherein the left part and the right part comprise tightening means to bring the left part and the right part closer to each other, and to tighten the left part and the right part on the spinous process by integrally connecting the left part and the right part,
wherein at least one of the left part and the right part delimits a reception recess for said longitudinal element, and wherein the linking element comprises holding means for holding the longitudinal element in said recess, and
wherein at least one of the left part and of the right part comprises immobilization means for the linking element that is assembled on the vertebra.

2. The linking element according to claim 1, in which the tightening means comprises locking devices, which are provided on the left part and on the right part.

3. The linking element according to claim 2, in which the tightening means comprise a first and a second pair of locking devices.

4. The linking element according to claim 3, in which the first locking means pair is arranged in proximity to the inferior side of the spinous process, and the second locking means pair is arranged in proximity to the superior side of the spinous process.

5. The linking element according to claim 4, in which a first pair of the linking element ensures an immobilization of the left part with the right part in translation along three mutually orthogonal directions, and wherein the second pair ensures an immobilization of the left part with the right part with a degree of freedom in translation.

6. The linking element according to claims 5, where the first pair comprises a serrated pin having rotational symmetry about its insertion axis (B) in a corresponding orifice, and the second pair comprises a toothed tongue, which is adapted for insertion into a corresponding clamp.

7. The linking element according to claim 6, wherein the immobilization means comprises an infra-laminary hook that is intended to become anchor to the infra-laminar part of said vertebra.

8. The linking element according to claim 7, where the immobilization means comprises a laminary cutting edge, that is intended to anchor to the cephalad part of the spinous process of the vertebra.

9. The linking element according to claim 8, comprising at least one bone growth orifice that opens opposite said vertebra and that traverses, a plurality of bone growth orifices distributed over the entire left part and right part.

10. The linking element according to claim 9, comprising at least one penetrating edge intended to penetrate said vertebra.

11. The linking element according to claim 10, characterized in that it comprises at least one pair of penetrating edges that define a bone growth furrow between themselves.

12. The linking element according to a claim 11, wherein the laminary surface is formed by a plurality of flexible legs.

13. A vertebral osteosynthesis device, comprising at least one longitudinal element and linking elements for linking said longitudinal element to at least two vertebrae.

14. The vertebral osteosynthesis device according to claim 13, including a left longitudinal element and a right longitudinal element that respectively extend on the left side and on the right side of the spinous process of the vertebra, and wherein said left part receives the left longitudinal element and said right part receives the right longitudinal element.

15. A method for implanting a vertebral osteosynthesis clerice on the spinal column, the method including:
providing a linking element located between longituional ellemrnt and a vertebra; providing a tightening means within said linking element to assist with mounting on a spinous process;
providing a holding means within said linking element for holding said longitudical element; and
providing an immobilization means to assist with immbolization of said linking element on the vertebra.

16. The method of claim 15, wherein said linking element includes a left linking part and a right linking part, wherein each part, respectively, extends on the left side and on the right side of the spinous process of the vertebra.

17. The method of claim 16, wherein said left linking part and said right linking part respectively, delimit a spinous surface shaped to rest on a the spinous process and a laminary surface shaped to rest on the posterior surface of the lamina of said vertebra.

18. The method of claim 16, wherein the left linking part and the right linking part comprise said tightening means that upon closing towards each other tighten on the spinous process.

19. The method of claim 16, wherein the left linking part and the right linking part delimit a reception recess for holding said longitudual element.

20. The method of claim 16, wherein the left linking part and the right linking part comprise said immobilization means.
